(19) United States
(12) Patent Application Publication
(10) Pub. No.: US 2012/0239055 A1
(43) Pub. Date: Sep. 20, 2012

(54) METHOD FOR TREATMENT OF THE FEMALE PELVIC FLOOR AND PERINEAL ORGANS WITH EXTRACORPOREAL SHOCKWAVES

(76) Inventors: Avner SPECTOR, Savyon (IL); Michal Jeshurun-Gutshtat, Elkana (IL); Nadav Dvir, Raanana (IL)

(21) Appl. No.: 13/411,669

(22) Filed: Mar. 5, 2012

Related U.S. Application Data
(60) Provisional application No. 61/449,129, filed on Mar. 4, 2011.

(51) Int. Cl. A61B 17/42

(52) U.S. Cl. 606/119

(57) ABSTRACT

The present invention relates to a method for treating the pelvic floor and perineal organs including female genitalia with extracorporeal shockwaves and in particular, to such a method for the treatment of the female pelvic floor and genitalia.

### CROSS REFERENCE TO RELATED APPLICATIONS

This application claims the benefit of U.S. Provisional Application Ser. No. 61/449,129, filed on 4 Mar. 2011, which is hereby incorporated by reference in its entirety.

### FIELD OF THE INVENTION

The present invention relates to a method for treating the pelvic floor and perineal organs including female genitalia with extracorporeal shockwaves and in particular, to such a method for the treatment of the female pelvic floor and genitalia.

### BACKGROUND OF THE INVENTION

Extracorporeal shockwave therapy (herein referred to as ‘ESWT’) is non-surgical, non-invasive treatment of medical conditions using acoustic shockwaves. First use of shockwave therapy in the early 1980's was utilized to fragment kidney stones termed shockwave lithotripsy. Continued development of shockwave treatment showed the possibility of stimulating bone formation, angiogenesis, as well as other orthopedic indications.

A shockwave is a form of acoustic energy resulting from phenomena that create a sudden intense change in pressure for example an explosion or lightning. The intense changes in pressure produce strong waves of energy that can travel through any elastic medium such as air, water, human soft tissue, or certain solid substances such as bone.

Acoustic shockwaves are primarily generated by three different methods, electrohydraulic (also referred to as spark gap), electromagnetic (also referred to as ‘EMSE’), and piezoelectric. Each method needs an apparatus to focus the generated shockwave so as to provide a focal point and/or focal zone for the treatment area. In the focal zone shockwaves produce much higher pressure impulses as compared with the zones outside of the focal zone.

Mechanical means for focusing each of these methods is generally realized with an appropriate arrangement of surfaces reflecting the wave toward the desired focal point and/or an appropriate arrangement of the generating devices.

Spark gap systems incorporate an electrode (spark plug), to initiate a shockwave, and ellipsoid to focus the shockwave. EMSE systems utilize an electromagnetic coil and an opposing metal membrane. Piezoelectric systems form acoustical waves by mounting piezoelectric crystals to a spherical surface to provide focus. Of the three systems, the spark gap system is generally preferred in the art for generating therapeutic shockwaves ESWT as it introduces more of the generated shockwave energy to the treatment target site.

In spark gap systems, high energy shockwaves are generated when electricity is applied to an electrode positioned in an ellipsoid immersed in treated water. When the electrical charge is fired, a small amount of water is vaporized at the tip of the electrode and a shockwave is produced. The shockwave ricochets from the side of an ellipsoid and converges at a focal point, which may then be transferred to the area to be treated.

In electromagnetic systems an electrical impulse is circulated in a coil. The coil produces an electromagnetic field that expels a metallic membrane to produce the mechanical impulse.

In piezoelectric systems ceramic material with piezoelectric characteristics is subjected to an electrical impulse. The electric impulse modifies the dimension of the ceramic material to generate the desired mechanical impulse. A focal point is attained by covering a concave spherical surface with piezoelectric ceramics converging at the center of the sphere.

The method of focusing the generated shockwave has been greatly described in the art for example in U.S. Pat. Nos. 5,174,280 and 5,058,569, 5,033,456, EP1591070 all of which are incorporated herein by reference as if fully set forth.

Medical use of shockwave therapy provides noninvasive means for treating a variety of anomalies such as kidney stones, chronic orthopedic inflammation healing, bone healing (osteogenesis), wound healing, revascularization, angiogenesis are well known and described in medical literature.

### SUMMARY OF THE INVENTION

There is an unmet need for, and it would be highly useful to have, a device and a method providing extracorporeal shockwave therapy (ESWT) for treating and improving conditions associated with the female genitalia, reproductive tract and female pelvic floor.

Within the context of this application the terms aqueous solution, aqueous medium, or aqueous environment may be used interchangeably to refer to an enclosure, opening, lumen, or space that is placed in an aqueous solution or mixture for example including but not limited to water, medicated water, ionized water, oil, gel, treated water or the like solution or mixture in a liquid state.

Within the context of this application the term extracorporeal shockwave therapy (ESWT) refers to shockwave therapy provided with all forms of shockwave generating device.

Within the context of this application the term shockwave treatment device refers to a device comprising a controller and/or computer and a shockwave treatment applicator as is known in the art. For example, a shockwave treatment device comprises controller and/or computer that controls the shockwave treatment produced by the shockwave treatment applicator.

Within the context of this application the terms female pelvic floor (herein referred to ‘FPF’), perineum, genitalia and reproductive tract may be used to refer to the muscle fibers of the levator anii and coccygeus and associated connective tissue which span the area underneath the pelvis, female reproductive tract and/or genitalia, and perineum an interchangeably refer to any of the anatomical structures associated with the female anatomy spanning and including the lower portion of the torso. The term female pelvic floor as referred to within this application may be interchangeably used to refer to any anatomical structure including but not limited to the following anatomical structures vulva which includes external genital organs of the female, including the labia majora, labia minora, clitoris, and vestibule of the vagina, vagina, anterior and posterior fornix, cervix, uterus, fundus, fallopian tube, fimbriae and ovaries. These terms may further refer to any epithelial, lymphatic, vascular and/or neural cells, tissue and/or structures that may be directly or indirectly associated with the anatomical structures of the female pelvic floor, for example as referenced above. For example associative structures of the female pelvic floor may for example include but is not limited to the internal pudendal artery, the internal pudendal vein, pudendal nerve, and superficial inguinal lymph node.

An optional embodiment of the present invention provides for a method for ESWT of the female pelvic floor, genitalia and reproductive tract. Optionally the ESWT of the female pelvic floor and/or reproductive tract may for example treat any condition, anomaly, disease, acute condition, chronic condition associated therewith for example including but not limited to urinary incontinence, uterus prolapse, pelvic organ prolapsed, Thin Endometrial Lining, Chronic Pelvic Pain Syndrome (herein interchangeably referred to as ‘CPPS’), Interstitial Cystitis, Urethral Syndrome, Non-Responsive Vaginal Infections, Recurrent spontaneous abortions, Uterine Receptivity, Vaginal Bleeding, cervical spotting, In Vitro Fertilization (herein interchangeably referred to as ‘IVF’), Vaginismus, vestibulitis, to Minimal Endometrial Injury, Increased Decidualization of Endometrium, Chronic infections of the genitourinary system, Chlamydial infection, uterine adnexa inflammation, Pelvic Inflammatory Disease (herein interchangeably referred to as ‘PID’), yeast infection (candidiasis), Female Sexual Dysfunction, (sexuality, sexual gratification, sexual arousal, female orgasm or the like condition for example due to primary and/or secondary physical pathogenesis), disease or anomaly associated with the female reproductive tract or the female pelvic floor region.

The method according to an optional embodiment of the present invention comprises associating a shockwave generating device within an aqueous environment to generate and treat the female pelvic floor and/or genital and/or perineum and/or reproductive tract within a desired focal zone. Optionally the shockwave generating device may be placed in a number of positions relative to the treatment area to generate and or produce the required focal zone.

Optionally the shockwave generating device may be associated with the female pelvic floor region directly or indirectly. Preferably the shockwave device is positioned so that it generates and/or produces a focal treatment zone comprising at least a portion of the female pelvic floor region being treated.

Optionally a noninvasive indirect approach to the female pelvic floor may be attained by placing and/or coupling a shockwave generating device about the skin surface near the female pelvic floor for example about the lower torso, for example below the naval and/or above the vagina. Optionally shockwave device may be placed on anterior and/or posterior and/or perineal skin surface about the lower torso. For example a noninvasive indirect approach may be used for treating CPPS, Interstitial Cystitis, Thin Endometrial Lining or the like.

Optionally a noninvasive direct approach to the female pelvic floor region may be attained by placing and/or coupling a shockwave generating device within the female pelvic floor region for example through the vaginal canal or anal canal. For example a vaginal approach may be utilized in providing treatment for at least one or more of Urethral Syndrome, Non-Responsive Vaginal Infections, Recurrent abortions, thin endometrial lininig, Vaginal Bleeding (‘Religious Purity Problems’), Vaginismus, female sexual dysfunction or the like.

Optionally an invasive approach to the female pelvic floor region may be attained by placing and/or coupling a shockwave generating device through a port, keyhole or incision providing a direct approach to the female pelvic floor region anatomy.

Optionally a desired focal treatment zone may be generated with at least one or more shockwave generating device utilizing at least one or more or a combination of direct, indirect, invasive or noninvasive approaches for example including but not limited to an anterior approach, posterior approach, perineal approach, vaginal canal, anal canal, incision, keyhole, port or any combination thereof.

Optionally, the method of treatment of the female pelvic floor and/or reproductive tract according to the present invention produces a shockwave regimen determined based on at least one or more parameters for example including but not limited to shockwave parameters, treatment protocol parameters, anatomical parameters, or the like.

Optionally protocol parameters for example including but not limited to the number of treatments sessions, the duration of a treatment protocol, timing of active and/or inactive treatment sessions, frequency of session, or the like.

Optionally the number of active treatment sessions may be provided from about 1 session to about 18 sessions. Optionally 12 active treatments may be provided during the treatment protocol according to the present invention. Optionally number of active treatment session may for example be 1, or 2, or 3, or 4 or 5 or 6, or 7 or 8 or 9 or 10 or 11, or 12 or 13 or 14 or 15 or 16 or 17 or 18 or 19 or 20 or so sessions.

Optionally the duration of the treatment protocol according to the present invention may be from about 1 day up to about 18 weeks or the equivalent of 1 day up to about 126 days. Optionally treatment may be provided periodically, continuously, sequentially, intermittently, according to a schedule comprising consecutive sessions and/or with at least one or more intersession recesses. Optionally the length of the recesses may vary according to the required treatment protocol.

Optionally, shockwave parameters may for example include but are not limited to number of shockwaves, frequency of shockwaves and intensity of the shockwave, or the like.

Optionally shockwave intensity may be provided from about 0.02 mJ/mm² to about 0.18 mJ/mm² Optionally and preferably a shockwave intensity may be provided at about 0.09 mJ/mm².

Optionally shockwave frequency may be provided from about 60 shockwaves per minute to about 360 shockwaves per minute. Optionally and preferably a shockwave frequency may be provided at about 120 shockwaves per minute.

Optionally the number of shockwaves per treatment session may be provided from about 100 shockwave up to about 5000 shockwaves. Optionally and preferably about 1800 shockwaves per session may be provided.

Unless otherwise defined, all technical and scientific terms used herein have the same meaning as commonly understood by one of ordinary skill in the art to which this invention belongs. The materials, methods, and examples provided herein are illustrative only and not intended to be limiting. Implementation of the method and system of the present invention involves performing or completing certain selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustrative diagram of optional device configurations that may be utilized to provide the treatment to the female pelvic floor region according to an optional embodiment of the present invention;

FIG. 2A-D are schematic illustrative diagram of optional approaches in producing a treatment focal zone within the female pelvic floor according to an optional embodiment of the present invention; and

FIG. 3 is a flowchart of an exemplary method according to the present invention for ESWT of the female pelvic floor.

### DESCRIPTION OF THE PREFERRED EMBODIMENTS

The principles and operation of the present invention may be better understood with reference to the drawings and the accompanying description.

The following figure reference labels are used throughout the description to refer to similarly functioning parts.

10 Female Pelvic Floor;

100 ESWT applicator—shockwave generator;

102 ultrasound imaging probe;

110 shockwave focal zone.

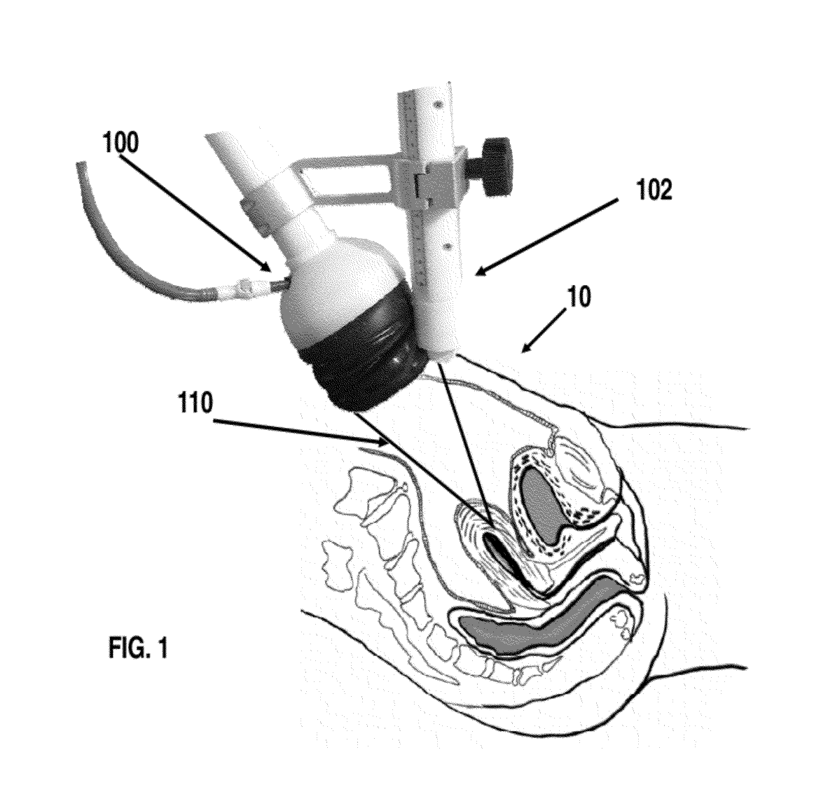

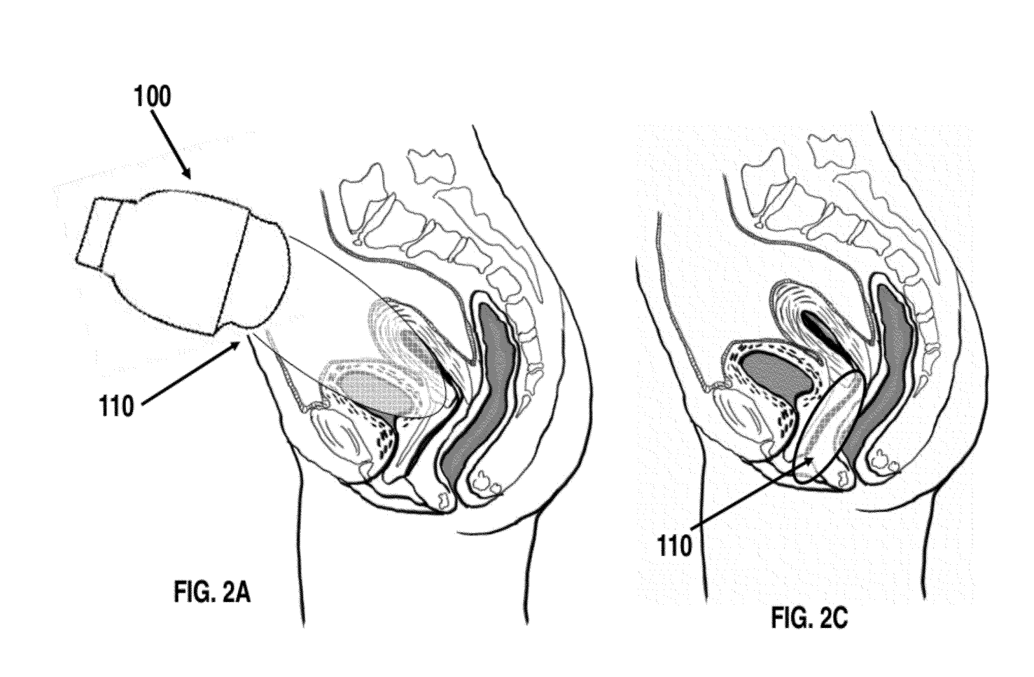

FIG. 1 provides an illustrative diagram of an optional treatment of the female pelvic floor region 10 according to an optional embodiment of the present invention and specifically treating the uterine epithelium with a shockwave treatment device 100 under the visual guidance of ultrasound probe 102 targeting a focal zone 110.

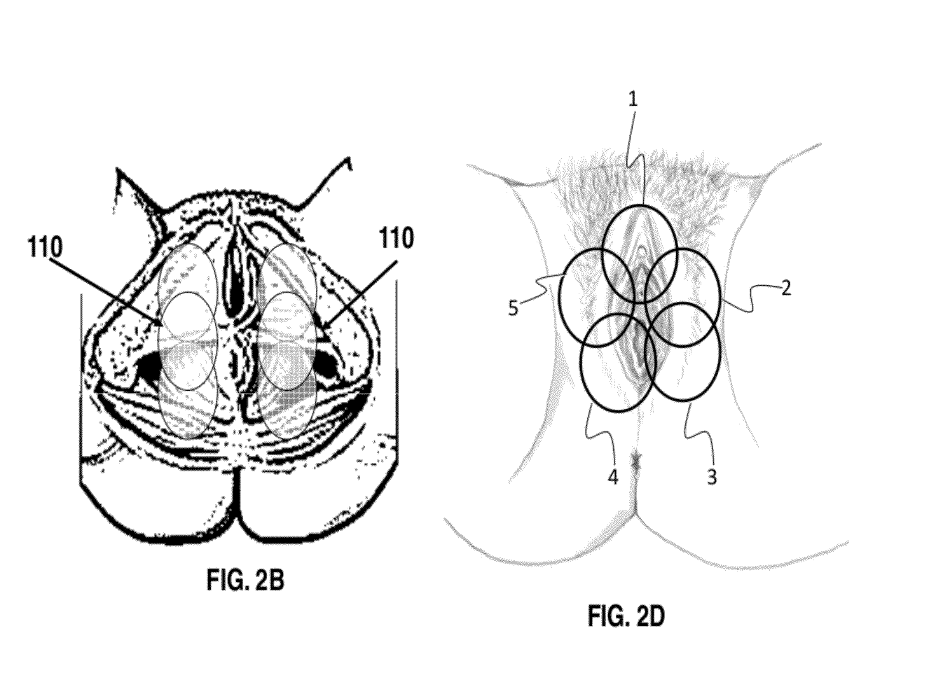

FIG. 2A-D provides optional illustrative views of the female pelvic floor region as well as varying focal zones 110 that may be used to treat different conditions associated with the female pelvic floor. FIGS. 2A-C further provide a depiction of different approaches that may be used to treat areas within the female pelvic floor. FIG. 2A depicts an anterior approach in treating a focal zone about the uterus and vaginal canal. FIGS. 2B and 2D depicts focal zones that may be treated with a perineal approach about the anus and vagina. FIG. 2C depicts focal zone that may be treated with a vaginal canal approach to directly treat the vagina and/or cervix.

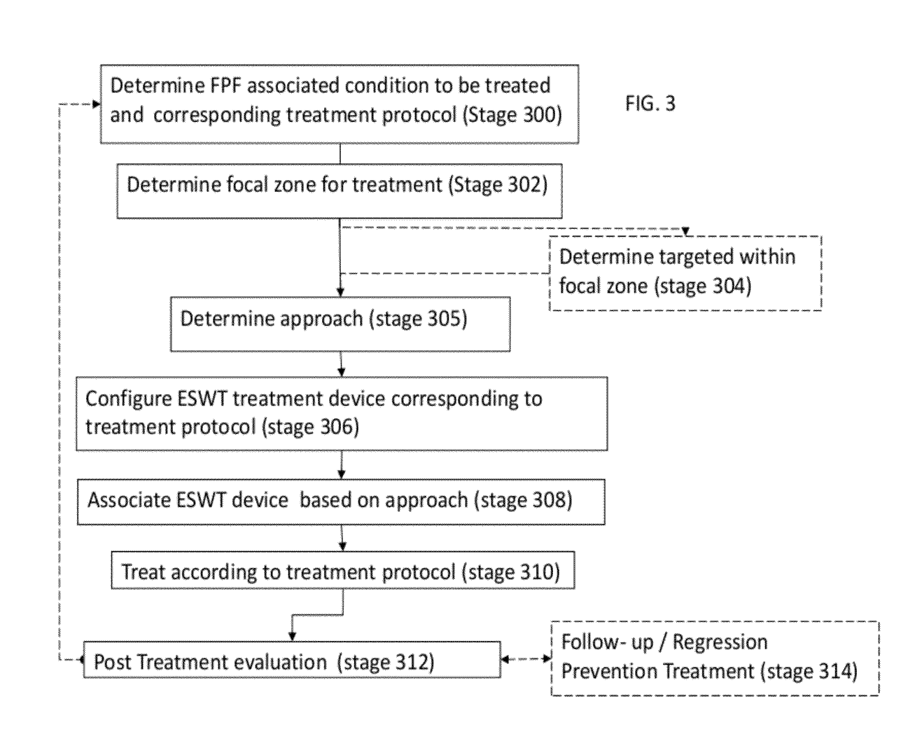

FIG. 3 shows a flowchart of an optional method of treatment of the Female Pelvic Floor according to an optional embodiment of the present invention. First in stage 300 at least one suspect condition, anomaly, disease, acute condition, chronic condition or the like associated with the female pelvic floor region is determined along with at least one or more treatment protocol for treating it. Next in stage 302, the ESWT treatment focal zone, for example 110 of FIGS. 1-2 is determined most preferably for providing optimal treatment results. Optionally, a localized and/or primary ESWT targeted zone with the focal zone, for example 110 of FIG. 2B, is determined in stage 304. Next in stage 305 the treatment approach is determined so as to provide the optimal focal treatment zone 110 determined in stages 302 and 304. Optionally at least one or more approaches for example including but not limited to direct, indirect, invasive or non-invasive, anterior, posterior, perineal, vaginal canal, anal canal alone or in combination may be selected so as to obtain the optimal focal zone 110. For example, a combination of an internal vaginal canal approach and an anterior abdominal surface approach may be utilized to treat a focal zone centered about the cervical end of the endometerium. For example, treatment of a focal zone centered about the fundal region of the endometrium may be attained with an anterior approach.

Next in stage 306, based on at least one or more of the condition being treated, the selected treatment protocol as determined in stage 300, the focal zone and approach as determined in stages 302-305, the treatment device ESWT 100 device is configured to provide appropriate treatment. Most preferably, ESWT device 100 is configured by adjusting software settings to provide the appropriate treatment protocol and focal zone. Next in stage 308, ESWT device 100 is associated with the female pelvic floor region according to at least one or more approaches determined in stage 305, for example as illustrated in FIG. 2A-C, thereafter treatment is provided in stage 310 as determined in stage 306.

Next in stage 312 treatment assessment and evaluation is provided for determining follow up treatment to undertake, optionally to provide further treatment with a new focal treatment zone, as described in stage 300, or to continue with follow up treatments in stage 314.

Optionally stage 314 provides for follow up treatments as an addition to the treatment protocol defined in 306. Optionally stage 314 may be used to provide for continued treatment, complimentary treatment, ongoing to preventative treatment or the like treatment in continuation of the treatment protocol of stage 306. Optionally following stage 314 treatment is reevaluated in stage 312.

Optionally treatment protocol determined in stage 306 comprises at least one or more parameters, for example including shockwave parameters, treatment protocol parameters, anatomical parameters or the like parameters. For example protocol parameters may include but are not limited to the number of treatment sessions, treatment protocol duration, period of active/inactive treatment sessions, duration of interval periods without ESWT, number of treatment zones, number of shockwaves per zone, total number of shockwaves, treatment intensity, treatment frequency, or the like parameters. Table 1 below summaries the optional treatment parameters and their optional value ranges for treating conditions, anomalies, and/or diseases associated with the female pelvic floor, reproductive tract and/or genitalia.

TABLE 1

| | Parameter | | |
|---|---|---|---|
| S.N. | Group | Parameter | Value Range |
| 1 | Protocol | Number of | 1-18 |
| | parameters | treatment sessions | |
| 2 | | Treatment Protocol | 1 day-18 weeks or the |
| | | Duration | equivalent of 1-126 days |
| 3 | | Weeks of active | Weeks 1-5 |
| | | Treatment sessions | |
| 4 | | Weeks of non- | Weeks 1-6 |
| | | treatment Interval | |
| 5 | Anatomical | Number of | 1-10 |
| | parameters | treatment zones | |
| 6 | Shockwave | Number of | 100-500 |
| | Parameters | Shockwaves per | |
| | | zone | |
| 7 | | Total number of | 100-5000 |
| | | shockwaves per | |
| | | session | |
| 8 | | Shockwave | 0.02-0.18 mJ/mm² |
| | | intensity | {50-200 bar} |
| 9 | | Shockwave | 60/min-360/min |
| | | Frequency | |

Optionally the shockwave parameters for example comprise parameters including but not limited to shockwave frequency, shockwave intensity and number of shockwaves delivered. Optionally and preferably the shockwave parameters are controllable and may be set in accordance with a particular treatment protocol, for example in stage 306 of FIG. 3. Optionally, shockwave parameters may be determined and defined based on the anatomical area being treated, as shown and described above in FIGS. 1-2.

Optionally shockwave parameters may be determined and defined according to at least one or more effective treatment sought for example including but not limited to vasodilatation, angiogenesis, enhanced endothelial function, neural regeneration, increase cytoplasmic calcium concentration, reduce cytoplasmic calcium concentration, enzymatic inhibition, enzymatic excitation, or the like taken alone or in any combination thereof

Optionally shockwave intensity provided during a single active treatment according to the present invention comprises shockwaves intensity of up to about 300 bar, more preferably from about 50 bar up to about 200 bar, more preferably about 100 bar, most preferably about 125 bar. Optionally the shockwave intensity provided during a single active treatment comprises shockwaves intensity of up to about 0.25 mJ/mm², more preferably from about 0.02 mJ/mm² up to about 0.18 mJ/mm², and most preferably at to about 0.09 mJ/mm² Optionally shockwave intensity parameters measurement may for example be provided in the unit measurements of bar and/or mJ/mm² or the like equivalent unit measurement for shockwave intensity as is known and accepted in the art.

Optionally shockwave frequency provided during a single active treatment according to the present invention comprises shockwaves frequency of up to about 300 shockwaves per minute, optionally from about 60 up to about 360 shockwaves per minute and optionally and preferably about 120 shockwaves per minute.

### EXAMPLE 1

### Treatment of Female Sexuality, Sexual Gratification, Arousal and/or Orgasm

An optional embodiment of the present invention provides for a treating conditions associated with female sexuality, sexual gratification, arousal and/or orgasm difficulties that may be present in women that suffer from vaginal atrophy, vaginal dryness and or dyspareunia, vaginal bleeding, pruritus vulvae, and urinary symptoms or the like conditions associated with female sexuality, arousal and/or orgasm. An optional treatment protocol may comprise of a combination of about 1500 shockwaves comprising a vaginal approach, as shown in FIG. 2D that may span a 9 week period. Optionally the treatment may include a plurality of focal zones, for example 10 focal zones. Optionally each foal zone may be treated with a plurality of shockwaves for example, each of the focal zones may be treated with 100 shockwaves and up to about 500 shockwaves per zone. Optionally the treatment Intensity is about 0.09 mJ/mm² to about 0.1 mJ/mm² with a frequency of about 120 shockwaves/min

Optionally the treatment may be spread over a 9 week period where weeks 1-3 and 7-9 include treatments twice a week while weeks 4-6 are devoid of shockwave treatment.

### EXAMPLE 2

### Treatment of CPPS

An optional embodiment of the present invention provides for a treating CPPS. The CPPS treatment protocol according to an optional embodiment of the present invention optionally and preferably comprises a combination of about 1800 shockwaves spanning two approaches including a perineal approach, as shown in FIG. 2B and an anterior approach as shown in FIG. 2A, and spanning a 9 week period. Optionally the treatment may include a plurality of focal zones for example 8 focal zones including 6 perineal treatment zones, and 2 anterior treatment zones as shown in FIGS. 2A-B. Optionally each focal zone may be treated with a plurality of shockwaves for example, each of the perineal focal zones may be treated with 200 shockwaves while each anterior focal zone may be treated with 300 shockwaves. Optionally the treatment Intensity is about 0.09 mJ/mm² to about 0.1 mJ/mm² with a frequency of about 120 shockwaves/min.

Optionally the treatment may be spread over a 9 week period where weeks 1-3 and 7-9 include treatments, optionally twice a week, while weeks 4-6 are devoid of shockwave treatment.

### EXAMPLE 3

### Treatment of Recurring Abortion or Lack of Pregnancy Due to Pathological Embryonic Implantation

An optional embodiment of the present invention provides for the treatment of recurring abortions or inability to attain pregnancy that may be due to pathological implantation, optionally with or without in-vitro fertilization (‘IVF’) treatments. Optionally and preferably treatment may be provided on day 1 or 2 of the menstrual cycle and prior to fertilization whether natural or otherwise assisted, for example by in-vitro fertilization (‘IVF’).

Timing of treatment may vary if treatment is accompanied by IVF or not. For example, if treatment is accompanied with IVF where the timing of embryo transfer (‘ET’) is controllable, the shockwave treatment may continue from day 1 or 2 of the menstrual cycle up until embryo transfer. Optionally and preferably the frequency of treatment is about 2 to 3 times a to week within the treatment window, prior to embryo transfer. Optionally treatment may be started on day 1 or 2 of menstrual cycle and administered on a daily basis, up to prior to fertilization.

If treatment is provided with unassisted fertilization and/or natural fertilization and/or non-IVF fertilization conception timing of treatment and/or the treatment window may for example span day 1 or 2 of the menstrual cycle and until ovulation. Optionally the treatment window may span day 1 or 2 of the menstrual cycle until the end of the cycle, for example on day 7. Optionally the length of treatment and/or treatment window may be correlated to the length of mensus cycle. For example, a 28 day cycle may optionally correspond to a treatment window of six (6) days while a 33 day cycle may optionally correspond to an eight (8) day treatment window.

Optionally the shockwave treatment intensity is about 0.09 mJ/mm² to about 0.1 mJ/mm² with a frequency of about 120 shockwaves/min Most preferably the focal zone is localized to the Endometrium-Myometrium Interface, with a trans-abdominal anterior approach, optionally an intravaginal approach as depicted in FIG. 2C may also be utilized. Optionally and preferably about 3 to about 5 focal zones are treated with 200 shockwaves delivered within each focal zone.

While the invention has been described with respect to a limited number of embodiments, it will be appreciated that many variations, modifications and other applications of the invention may be made.

What is claimed is:

1. A method for the treatment of acute condition, chronic condition, conditions, diseases or anomalies associated with the Female Pelvic Floor the method comprising:
a. determining a targeted area within the female pelvic floor region to be treated;
b. associating a shockwave generating device within an aqueous environment able to treat said targeted area; and
c. applying a shockwave regimen to said treatment area of the female pelvic floor wherein said shockwave device produces a focal zone comprising at least a portion of said targeted area.

2. The method of claim 1 wherein said focal zone includes at least one or more anatomical structures of the female pelvic floor selected from the group consisting of vagina, vulva, labia majora, labia minora, clitoris, vestibule, fornix, cervix, uterus, fundus, fallopian tube, fimbriae, ovaries and any portion thereof.

3. The method of claim 2 wherein said shockwave treatment is applied to any epithelial, lymphatic, vascular and/or neural structures directly or indirectly associated with said anatomical structures.

4. The method of claim 3 wherein the vascular structures are selected from one or more of internal pudendal artery, internal pudendal vein, pudendal nerve, and Superficial inguinal lymph node, or any portion thereof.

5. The method of claim 1 wherein said shockwave regimen produce a treatment regimen determined based on at least one parameter chosen from the group consisting of shockwave parameters, treatment protocol parameters, and anatomical parameters.

6. The method of claim 5 wherein said shockwave parameters comprise number of shockwaves, frequency of shockwaves and intensity of said shockwave.

7. The method of claim 6 wherein said shockwave intensity is about from about 50 bar to about 200 bar.

8. The method of claim 6 wherein said shockwave frequency is from about 60 to about 360 shockwaves per min.

9. The method of claim 6 wherein said number of shockwaves is up to about 5000.

10. The method of claim 1 further comprising coupling said shockwave regimen with a drug or medicinal treatment.

11. The method of claim 10 wherein said medicament or drug is chosen form the group consisting of stem cells, growth factors, hormones, peptides, biologics, DNA, RNA, animal extract, plant extract, oil, gel, balm, cream.

12. The method of claim 1 wherein said condition, anomaly, disease, acute condition, chronic condition is selected from the group consisting of urinary incontinence, uterus prolapse, pelvic organ prolapsed, thin endometrial lining, chronic pelvic pain syndrome (CPPS), interstitial cystitis, urethral syndrome, non-responsive vaginal infections, recurrent spontaneous abortions, uterine receptivity, vaginal bleeding, cervical spotting, in vitro fertilization (IVF), Female Sexual Dysfunction, vaginismus, vestibulitis, minimal endometrial injury, increased decidualization of endometrium, chronic infections, vaginitis, Chlamydia, uterine adnexa inflammation, pelvic inflammatory disease, yeast infection (candidiasis), sexuality, sexual arousal, inability to reach orgasm, sexual gratification, or any combination thereof.

13. A method for the treatment of a female subject for the improvement of said subject's ability to participate in and/or have sexual intercourse by applying a regimen of shockwave therapy to at least one or more focal zones within the Female Pelvic Floor anatomy.

14. The method of claim 13 wherein said treatment improves said subject's ability to experience sexual arousal and/or reach orgasm and/or to reach sexual gratification.
